(19) United States
(12) Patent Application Publication
(10) Pub. No.: US 2006/0029980 A1
(43) Pub. Date: Feb. 9, 2006

(54) Method for diagnosing obstructive sleep apnea

(76) Inventors: David Gozal, Louisville, KY (US); Saeed A. Jortani, Louisville, KY (US); Roland Valdes JR., Simpsonville, KY (US)

Correspondence Address: STITES & HARBISON, PLLC, 400 W MARKET ST, SUITE 1800, LOUISVILLE, KY, 40202-3352, US

(21) Appl. No.: 11/199,355

(22) Filed: Aug. 8, 2005

Related U.S. Application Data
(60) Provisional application No. 60/599,930, filed on Aug. 9, 2004.

(51) Int. Cl. G01N 33/53

(52) U.S. Cl. 435/7.1

(57) ABSTRACT

A method for diagnosing obstructive sleep apnea in a patient comprises identifying at least one protein biomarker for obstructive sleep apnea; obtaining a sample from the patient; and testing the sample for presence of the at least one protein biomarker. The protein biomarkers may include alpha-1 B-glycoprotein; kallikrein, laminin, aldosterone-binding protein and/or urocortin-2 precursor. The presence of the protein biomarkers may be detected using antibodies. These antibodies may be provided in an array for detecting the presence of the at least one protein biomarker or a pattern of protein biomarkers.

### CROSS REFERENCES TO RELATED APPLICATIONS

This application claims priority from U.S. Provisional Application Ser. No. 60/599,930 filed Aug. 9, 2004, the entire disclosure of which is incorporated herein by this reference.

### FIELD OF THE INVENTION

The present invention relates generally to diagnosis of sleep apnea and, more particularly, to methods for diagnosing obstructive sleep apnea.

### BACKGROUND OF THE INVENTION

Obstructive sleep apnea (OSA) is a breathing disorder characterized by repeated events of partial or complete obstruction of the upper airways during sleep, leading to recurring episodes of hypercapnia, hypoxemia, and arousal throughout the night for the purpose of recommencing breathing. Obstruction of the airway is caused in a variety of manners; for example, the tonsils or adenoids may become large enough, relative to the airway size, to cause or contribute to a blockage of air flow through the airway. Obstructive Sleep Apnea is a frequent condition affecting up to 3-5% of children and adults and imposes substantial neurocognitive, psychological, metabolic, and cardiovascular morbidities.

Patients who snore, but who do not have gas exchange abnormalities or evidence of snore-associated alterations in sleep architecture, are considered to have primary snoring (PS). PS is much more prevalent than OSA with a PS:OSA ratio of 2-5 depending on the age of the patients and multiple other considerations. However, in the clinical setting, diagnosing OSA by conducting physical examinations or studying family and patient history has been largely unsuccessful because such methods have an extremely poor predictive value in differentiating between PS and OSA.

Thus, current diagnostic approaches for OSA require an overnight sleep study, which is costly, inconvenient, and labor-intensive. Furthermore, the relative unavailability of suitable sleep diagnostic facilities leads to long waiting periods and unnecessary delays in diagnosis and treatment.

Accordingly, there remains a need in the art for a method which satisfactorily addresses the above-identified problems.

### SUMMARY OF THE INVENTION

The present invention meets the above identified needs, and others, by providing a method for diagnosing obstructive sleep apnea (OSA) using one or more non-invasive biomarkers, which method is capable of reliably distinguishing between OSA and primary snoring (PS). The method detects identifies protein biomarkers that are specific to OSA in a sample collected from a patient, for example, a urine or serum sample.

An exemplary method of the present invention includes: identifying at least one protein biomarker for obstructive sleep apnea; obtaining a sample of from the patient; and testing the sample for presence of the at least one protein biomarker or a pattern of protein biomarkers.

Another exemplary method of the present invention includes: providing antibodies to one or more protein biomarkers; obtaining a sample from the patient; incubating the antibodies and the sample; and detecting binding of the antibodies and proteins in the sample.

Protein biomarkers may be identified by various methods, for example, by using of mass spectrometry and data mining approaches. Protein biomarkers may have molecular weights that are less than about 8,500 Da, that range from about 2000 to about 5000 Da, or that range from about 2,350 to about 2,643 Da.

Examples of identified protein biomarkers include: alpha-1B-glycoprotein, kallikrein, laminin, aldosterone-binding protein, and urocortin-2 precursor (urocortin II, UcnII, stresscopin-related peptide, urcortin-related peptide).

### DESCRIPTION OF THE DRAWINGS

FIG. 1 is a flow chart depicting steps in an exemplary method of the present invention;

FIG. 2 is a decision tree analysis of serum samples collected from patients;

FIG. 3A is an averaged two-dimensional gel in patients without OSA (control), with arrows indicating differences in spots between the control and OSA;

FIG. 3B is an averaged two-dimensional gel in patients with OSA, with arrows indicating differences in spots between the control and OSA; and

FIG. 4 is a comparison of two mass spectra, the upper spectrum for low molecular weight proteins in urine of a patient with OSA, and the lower spectrum for low molecular weight proteins in urine of a patient with PS.

### DETAILED DESCRIPTION OF THE INVENTION

The present invention is a method for diagnosing obstructive sleep apnea (OSA) using one or more non-invasive biomarkers, which method is capable of reliably distinguishing between OSA and primary snoring (PS). Specifically, OSA and PS are associated with different proteomic profiles, allowing for the identification of protein biomarkers that reliably screen and allocate any snoring individual to the correct diagnostic category, whether it be OSA or primary habitual snoring without OSA. Thus, by detecting the protein biomarkers in a sample collected from the patient, a diagnosis can be made.

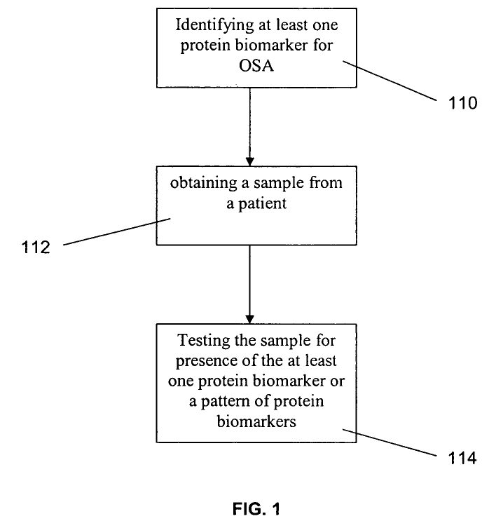

With reference to FIG. 1, an exemplary method of the present invention for diagnosing obstructive sleep apnea in a patient includes: identifying at least one protein biomarker for obstructive sleep apnea 110; obtaining a sample from the patient 112; and testing the sample for presence of the at least one protein biomarker or a pattern of protein biomarkers 114.

Another exemplary method of the present invention includes: providing antibodies to one or more protein biomarkers for obstructive sleep apnea; obtaining a sample from the patient; incubating the antibodies and the sample; and detecting binding of the antibodies and proteins in the sample.

Protein biomarkers may be identified by various methods, for example, by using of mass spectrometry and data mining approaches. Protein biomarkers may have molecular weights ranging from about 2,350 to about 2,643 Da, which proteins allow for accurate identification of OSA with about 75 to about 85% sensitivity and specificity. Protein biomarkers may also have molecular weights ranging from about 2,000 to about 5,000 Da. Protein biomarkers may also have molecular weights that are less than about 8,500 Da. Examples of identified protein biomarkers include: alpha-1B-glycoprotein, kallikrein, laminin, aldosterone-binding protein, and Urocortin-2 precursor (Urocortin II, UcnII, Stresscopin-related peptide, Urcortin-related peptide).

Body fluids may be used obtained from the patient for use as a sample in the method of the present invention, for example, first morning voided urine samples or serum samples.

The presence of one or more protein biomarkers or a pattern of protein biomarkers may be measured in a variety of manners, for example, the protein biomarkers may be detected using antibodies generated for the protein biomarkers and In situ calorimetric detection tests may then be conducted. For other examples, the protein biomarkers may be detected by automated immunoassays, mass-spectrometry, gel-based screening, point-of-care testing formats, or other wide-scale screening programs. For yet another example, antibodies or proteins may be immobilized on a substrate to create an antibody array or chip or a protein array or chip that may be provided for detecting the protein biomarkers.

The present invention is further illustrated by the following specific but non-limiting examples. The following examples are prophetic, notwithstanding the numerical values, results and/or data referred to and contained in the examples.

### EXAMPLES

### Protein Profiling and Biomarker Determination

A combination of mass spectrometry and data mining approaches results in an effective tool for biomarker discovery. See Wright G L Jr., Expert Review of Molecular Diagnostics 2(6):549-63 (2002), which is incorporated herein by this reference. Data mining is an automated or semi-automated search for relationships and global patterning within large body of data. Data mining techniques include data visualization and the use of algorithms. In supervised data mining, dependent variables are present; in unsupervised data mining, dependent variables are absent.

Surface Enhanced Laser Desorption/Ionization (SELDI) mass spectrometry, in combination with data mining, has been effectively used to discover biomarkers for ovarian, breast, prostate, lung, and other types of cancers, as well as, infectious diseases, neurological illness, and assessment of allograft rejection in renal transplantation. See Petricoin E F, et al., Lancet 359(9306):572-7 (2002); Li J, et al. Clinical Chemistry 48(8):1296-304 (2002); Qu Y, et al., Clinical Chemistry 48(10):1835-43, (2002); Zhang L, et al., Science 298(5595):995-1000 (2002); Vehmas A K, et al., DNA & Cell Biology 20(11):713-21 (2001); Clarke W et al., Annals of Surgery 237(5):660-4 (2003), each of which are incorporated herein by this reference.

A similar method using SELDI mass spectrometry, in combination with data mining, is used to identify protein biomarkers for OSA or primary habitual snoring without OSA. SELDI mass spectrometry based protein biomarker discovery allows for analyte capture, purification, analysis, and processing from complex biological mixtures to be performed directly on ProteinChip Array surfaces. In any event, urine or serum is collected from patients of interest, and their protein profiles are determined. A data mining approach using an algorithm known as the Classification and Regression Tree (CART) algorithm is used to identify biomarkers capable of diagnosing OSA or primary habitual snoring without OSA with both high clinical sensitivity and specificity.

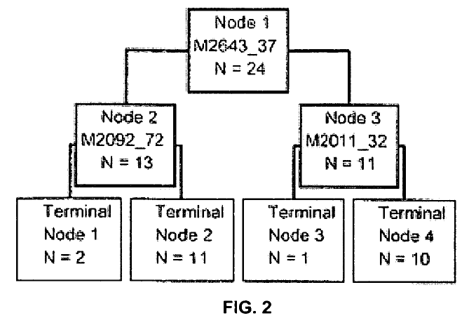

The Classification and Regression Tree (CART) algorithm is a hierarchical method for partitioning data into increasingly more homogenous groups. CART splits the data at each node in a decision tree using a rule which is selected to maximize the homogeneity of the two resultant groups. Generally, the rule at each splitting node is selected based on the data present, i.e., the data drives selection of the rule. FIG. 2 depicts a decision tree analysis of serum samples collected from 24 children, 12 controls and 12 OSA.

Detection of purified proteins is performed by laser desorption ionization time-of-flight mass analysis. Chemical and biochemical processing may be included at any step throughout the SELDI process to enhance the knowledge gained from a set of experiments. Subsequent data mining algorithm may be applied to discover profiles or signatures of proteins consistent with presence or absence of a disease or condition of interest. See Issaq H J, et al., Biochemical & Biophysical Research Communications 292(3):587-92 (2002), which is incorporated herein by this reference.

### Application of Protein-Chip Array Technology to Obstructive Sleep Apnea

Serum Proteomic Patterns Associated with OSA in Children

To determine whether sleep alterations associated with OSA condition leads to modification of a restricted number of identifiable proteins detectable in serum, the following study is conducted. Unfractionated sera is collected from about 20 children with OSA and about 20 children with PS and analyzed using SELDI technology (Ciphergen Biosystems, Fremont, Calif.) using different chip surface types, including: weak cation exchange (WCX) with low stringency (pH 4), metal binding (IMAC-Cu²⁺), strong cation exchange (SAX), and hydrophobic (H4) chips. Alpha-cyano-4-hydroxy cinnamic acid (CHCA) is used as energy-absorbing material (EAM) for each chip type. Normalized peaks are detected using the automated Ciphergen system and analyzed by both supervised (Biomarker Wizard Software, Ciphergen Biosystems, Inc., Fremont, Calif.) and unsupervised approaches (BPS—Biomarker Pattern Software, Ciphergen Biosystems, Inc., Fremont, Calif.).

Using the supervised software with decision tree analysis, about 12 cases in the PS and about 12 in the OSA group are used as the training set, and the remaining about 8 in each group are used to test the performance of the proteomic pattern which involves several proteins. Several low molecular weight proteins are discovered having molecular weights ranging from about 2,350 to about 2,643 Da, which proteins allow for accurate identification of OSA with about 75 to about 85% sensitivity and specificity. See, Gozal, et al., Abstract. Am. J. Respir. Crit. Care Med. 169:A715 (2004). In other studies, proteins having molecular weights less than about 5,000 Da are discovered. In other studies, proteins having molecular weights less than about 8,500 Da are discovered.

Identification of each or a group of proteins in the serum proteomic pattern is useful in developing antibody-based assays for further validation studies of useful biomarkers.

Differential Urinary Protein Expression in Patients with OSA

Adult male subjects referred for suspected OSA undergo overnight polysomnography, and apnea-hypopnea index (AHI) measurements are taken. AHI is a measure of the number of apneic and hypopneic episodes combined per hour of sleep. An apneic episode is generally considered a cessation of breathing while a hypopneic episode is generally considered an abnormal decrease in the depth and rate of breathing. The subjects are considered to have OSA if their AHI is greater than about 30 and are assigned to the control group if their AHI is less than about 5.

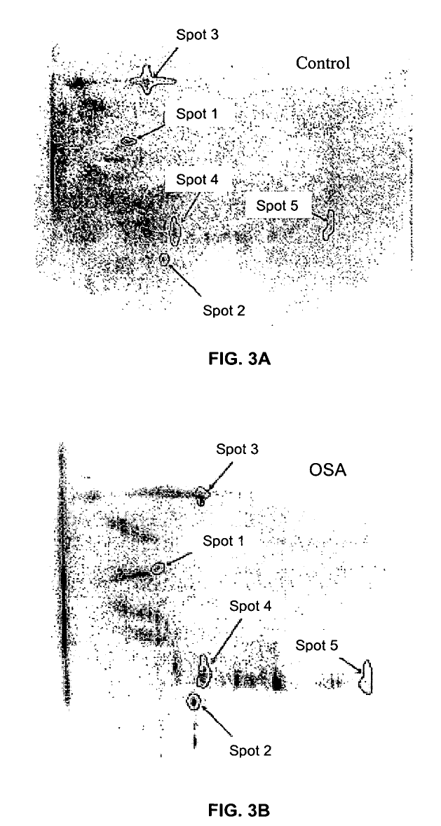

Urine samples are collected from about 3 control subjects and about 5 patients with OSA in the morning after the sleep study. Proteins are isolated by acetone precipitation and separated by two-dimensional polyacrylamide gel electrophoresis (2D-PAGE). Matrix-assisted laser desorption ionization-time-of-flight (MALDI-TOF) mass spectrometry followed by peptide mass fingerprinting are used for identification of separated proteins of interest. Out of about 67 total proteins previously identified in the human urinary proteome, a protein (alpha-1B-glycoprotein) is identified as being distinctly and consistently over-excreted in patients with OSA compared to controls. Urinary levels of this protein are about 19958±7554 densitometry units (DU) in OSA patients versus about 2252±402 DU in controls (p<0.03) suggesting that some degree of glomerular and/or tubular insult has occurred in these patients. The above-described study is repeated in about 5 children with obstructive sleep apnea and about 5 control children, and similar results are found. With reference to FIGS. 3A and 3B, which are averaged two-dimensional gel in control patients and patients with OSA, respectively, spots 1, 2, 3, 4 and 5 are differentially expressed in OSA children compared to controls. These proteins are identified using MALDI-TOF and include alpha-1B-glycoprotein, as well as kallikrein, laminin, and aldosterone-binding protein. See Gozal, et al. Abstract. Sleep (2003), which is incorporated herein by this reference. In other studies, urocortin-2-precursor is identified as a protein biomarker.

Urine Proteomic Patterns Associated with OSA in Children

To assess the feasibility of SELDI technology to discover proteomic patterns in urine that are capable of diagnosing OSA in children with potentially enhanced diagnostic sensitivity, the following study is conducted. About 23 children [6 controls, 9 PS (AHI<1), and 8 OSA (AHI>5)] as diagnosed by an overnight polysomnography are selected. Unfractionated first morning void urines from these subjects are directly loaded on different SELDI chip surface types, including: weak cation exchange (WCX) with low stringency (pH 4), metal binding (IMAC-Cu²⁺), and strong cation exchange (SAX). Alpha-cyano-4-hydroxy cinnamic acid (CHCA) is used as energy-absorbing material.

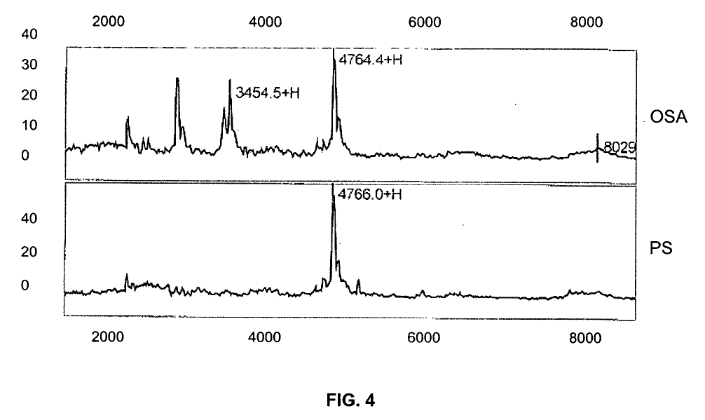

Normalized peaks are detected using the automated Ciphergen System and analyzed by both supervised (Biomarker Wizard, Ciphergen Biosystems, Inc., Fremont, Calif.) and unsupervised approaches (BPS—Biomarker Pattern Software, Ciphergen Biosystems, Inc., Fremont, Calif.). Pediatric patients with OSA express unique proteins in their urine that allow for separation of PS and OSA using simple cluster analyses. For example, with reference to FIG. 4, showing mass spectra of proteins in individual urine samples as derived from IMAC-Cu²⁺ chips, differences in proteins at certain molecular weights between PS and OSA samples are displayed. Individual peaks at particular molecular weights are different between PS and OSA sample groups (p<0.02).

### Application of Protein Profiles and Identified Biomarkers

The protein profiles are obtained and protein biomarkers are identified for OSA and/or primary habitual snoring without OSA, as described above. This information is then used to diagnose obstructive sleep apnea in a patient. For example, protein profiles are obtained from control patients and patients diagnosed by an overnight polysomnography as having OSA. These profiles are used to identify a protein biomarker for OSA, e.g., alpha-1B-glycoprotein and/or urocortin-2-precursor.

A urine sample is obtained from a patient who has not been diagnosed for OSA. The sample is tested for presence of the protein biomarker. The presence of the protein biomarker may be tested, for example, using antibodies generated for the protein biomarkers and an in situ colorimetric detection test.

Anther references that includes relevant information is Thongboonkerd, et al., J. Biol. Chem. 2002; 277:34708-34716, which is incorporated herein by this reference.

It will be apparent to those skilled in the art that various modifications and variations can be made in the present invention without departing from the scope or spirit of the invention. It is intended that the Specification and Examples be considered as exemplary only, and not intended to limit the scope and spirit of the invention.

Unless otherwise indicated, all numbers expressing quantities of ingredients, properties such as reaction conditions, and so forth used in the Specification and Sample Claims are to be understood as being modified in all instances by the term “about.” Accordingly, unless indicated to the contrary, the numerical parameters set forth in the Specification and Sample Claims are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references are incorporated herein by reference.

What is claimed is:

1. A method for diagnosing obstructive sleep apnea in a patient, comprising:
identifying at least one protein biomarker for obstructive sleep apnea;
obtaining a sample from the patient; and
testing the sample for presence of the at least one protein biomarker.

2. The method for diagnosing obstructive sleep apnea of claim 1, wherein the at least one protein biomarker has a molecular weight of less than about 8500 Da.

3. The method for diagnosing obstructive sleep apnea of claim 2, wherein the at least one protein biomarker has a molecular weight less of than about 5000 Da.

4. The method for diagnosing obstructive sleep apnea of claim 3, wherein the at least one protein biomarker has a molecular weight between about 2300 and about 2700 Da.

5. The method for diagnosing obstructive sleep apnea of claim 1, wherein the at least one protein biomarker is selected from the group consisting of: alpha-1B-glycoprotein; kallikrein, laminin, aldosterone-binding protein; and urocortin-2 precursor.

6. The method for diagnosing obstructive sleep apnea of claim 1, wherein the sample is a serum sample or a urine sample.

7. The method for diagnosing obstructive sleep apnea of claim 1 and further comprising: identifying a pattern of protein biomarkers for obstructive sleep apnea and testing the sample for the presence of the pattern of protein biomarkers.

8. A method for diagnosing obstructive sleep apnea in a patient, comprising:
providing antibodies to at least one protein biomarker;
obtaining a sample from the patient;
incubating the antibodies and the sample; and
detecting binding of the antibodies and proteins in the sample.

9. The method for diagnosing obstructive sleep apnea of claim 8, wherein at least one of the antibodies is selected from the group consisting of antibodies to: antibodies to alpha-1B-glycoprotein; antibodies to kallikrein; antibodies to laminin; antibodies to aldosterone-binding protein; and antibodies to urocortin-2 precursor.

10. The method for diagnosing obstructive sleep apnea of claim 8, wherein the antibodies are provided in an array.

11. The method for diagnosing obstructive sleep apnea of claim 8, wherein the sample is a serum sample or a urine sample.

12. The method for diagnosing obstructive sleep apnea of claim 8 and further comprising: identifying a pattern of protein biomarkers for obstructive sleep apnea and testing the sample for the presence of the pattern of protein biomarkers.

13. The method for diagnosing obstructive sleep apnea of claim 10 and further comprising: identifying a pattern of protein biomarkers for obstructive sleep apnea and testing the sample for the presence of the pattern of protein biomarkers.

14. The method for diagnosing obstructive sleep apnea of claim 8 and further comprising: providing a standard pattern of protein biomarkers generated using samples collected from individuals known to suffer from obstructive sleep apnea; and comparing the standard pattern to a pattern of protein biomarkers generated using the sample from the patient.
